(19) United States
(12) Patent Application Publication
(10) Pub. No.: US 2003/0186251 A1
(43) Pub. Date: Oct. 2, 2003

(54) Genome sequence tags

(75) Inventors: John J. Dunn, Bellport, NY (US); Daniel Van der Lelie, Shoreham, NY (US); Maureen K. Krause, Quogue, NY (US)

Correspondence Address: BROOKHAVEN SCIENCE ASSOCIATES/, BROOKHAVEN NATIONAL LABORATORY, BLDG. 475D - P.O. BOX 5000, UPTON, NY, 11973, US

(73) Assignee: Brookhaven Science Associates, LLC, Upton, NY (US)

(21) Appl. No.: 10/113,916

(22) Filed: Apr. 1, 2002

(51) Int. Cl. C12Q 1/68; C12P 19/34

(52) U.S. Cl. 435/6; 435/91.2

(57) ABSTRACT

Genomic Signature Tags (GSTs) are the products of a method for identifying and quantitatively analyzing genomic DNAs. The DNA is initially fragmented with a type II restriction enzyme. An oligonucleotide adapter containing a recognition site for MmeI, a type IIS restriction enzyme, is then used to release 21 bp tags from fixed positions in the DNA relative to the sites recognized by the fragmenting enzyme. These tags are PCR-amplified, purified, concatenated into longer molecules, and then cloned and sequenced. The tag sequences and abundances are used to create a GST profile that can identify and quantify the genome of origin within any complex DNA isolate. The total number of GSTs generated from a sample is determined by the incidence of recognition sites for the initial fragmenting enzyme.

### BACKGROUND OF THE INVENTION

Research toward improving the ability to detect and identify microbial genomes has risen to prominence because of its application to defense against bioterrorism and biological warfare. The steadily rising numbers of sequenced microbial genomes is also giving impetus to studies of natural populations in soil and water, with a view to understanding community composition and dynamics. In both scenarios, genomic information needs to be sufficiently detailed to distinguish among strains, and needs to provide a quantitative measure of the abundance of individual prokaryotic and eukaryotic genomes in an environmental DNA sample.

In the last twenty years, a variety of DNA-based techniques have been developed to allow comparisons of whole genomes. Perhaps one of the simplest approaches involves electrophoretic separation in two dimensions to separate restriction fragments. Fischer et al. (Cell 16: 191-200 (1979)) combined size separation in the first dimension with mobility in a denaturating gradient in the second dimension, to effectively separate and then probe whole-genome restriction digests. Restriction landmark genome scanning (RLGS) is a related method in which genomic DNA is end-labeled at sites generated by cleavage with a rare-cutting restriction enzyme, followed by gel electrophoretic size separation. The fragments are cleaved in situ with a second, more frequently cutting restriction enzyme and subjected to second-dimension electrophoresis to resolve the end-labeled fragments. A PCR-based method to generate fingerprint profiles of bacterial DNA by amplifying fragments generated by cutting at rare restriction sites has been developed (Masny et al., Biotechniques 31: 930-6 (2001)), but utility is limited to analysis of relatively small fragments. Recently, Rouillard et al. (Genome Res 11: 1453-9 (2001)) developed a software tool designated virtual genome scan (VGS), that makes it possible to predict automatically the sequence of first dimension NotI or EcoRV fragments, and second dimension HinfI or DpnII fragments in RLGS patterns of total human DNA, by matching fragment mobilities to those predicted from the draft human genome sequence. The utility of this method was demonstrated by its ability to identify a specific NotI-EcoRV fragment from human chromosome −1 that is frequently absent from restriction digests of neuroblastoma cells. Sequence prediction by VGS, as well as cloning of the fragment, showed that it contained a CpG island that is part of the human orthologue of the hamster homeobox gene Alx3 (Wimmer et al., Genes Chromosomes Cancer 33: 285-94 (2002)).

While VGS can provide a limited global survey for the presence or absence of a particular DNA fragment, it cannot directly identify novel sequences. VGS can be viewed as a closed architecture technique since it is inherently retrospective, relying on preestablished sequence information. Seeking a comprehensive DNA-based method for the identification and quantitation of microorganisms isolated from natural habitats, a RLGS approach with Serial Analyses of Gene Expression (SAGE) principles were combined to create an open architecture technique that provides Genomic Signature Tags (GSTs).

The SAGE technique is a powerful method for comprehensive analysis of gene expression patterns (Velculescu et al., Science 270: 484-7 (1995); Yu et al., Proc Natl Acad Sci U S A 96: 14517-22 (1999); and Zhang et al., Science 276: 1268-72 (1997)). In the original SAGE procedure, double-stranded cDNA is synthesized from poly (A)⁺ mRNA by priming first-strand cDNA synthesis with a biotinylated oligo (dT),₁₈ primer. The cDNA is then cut with a restriction endonuclease having a 4-bp recognition sequence (typically NlaIII, recognition sequence CATG, which theoretically results in cleavage on average every 256 bp), and the 31-terminal cDNA fragments are captured on streptavidin-coated magnetic beads. These fragments are ligated with DNA cassettes containing a recognition sequence for BsmFI, a type IIS restriction endonuclease. Subsequent cleavage with the BsmFI releases short (13-14 bp) but positionally defined sequences, referred to as tags, which are eventually concatenated into arrays and cloned into a plasmid vector for DNA sequencing. The power of the method is that many SAGE tags can be read serially from each clone during the sequencing step which vastly increases throughput. Over six million cDNA tags have been analyzed by this method since it was first described, many of which are publicly available at (http://www.ncbi.nlm.nih.gov/SAGE).

Recently, several groups have begun to use type IIS enzymes which cleave further into the cDNA than BsmFI in order to increase tag length. Longer tags are particularly useful in characterizing expression patterns in the absence of complete genome sequence data, i.e. from “uncharted transcriptomes”, and in designing primers to obtain full-cDNAs from transcripts whose tags are not currently present in RefSeq or similar expression databases. One very useful enzyme is MmeI which cleaves 20/18 bases past its non-palindromic (TCCRAC) recognition sequence (Boyd et al., Nucleic Acids Res 14: 5255-74 (1986); Tucholski et al., Gene 157: 87-92 (1995)). This length suggested that MmeI could be used to obtain unique tags directly from total microbial DNA since there are 421 or more than 4 trillion possible 21-mer tag sequences, which by far exceeds the number of 21-mers in most microbial genomes. Consequently, a MmeI tag should, in most cases, be able to uniquely identify its DNA source even in the absence of positional information. The commercial availability of MmeI prompted development of GSTs which, like RLGS, involves digestion of the DNA with two different restriction enzymes. After the digestion with the first enzyme the ends are biotinylated to allow their affinity capture after treatment with the second enzyme. A linker containing a MmeI recognition site is used to help liberate 21-bp tag sequences from the free ends of the captured fragments. The released monomeric tags are randomly ligated on themselves prior to cloning. The resulting sequences are identified through database matches or used to create a new database that is specific for a particular DNA sample. Using Yersinia pestis as a model system, it is demonstrated that the basic GST procedure can not only identify the DNA source, but also can pinpoint areas of a genome that might have undergone changes that add or delete restriction sites. It is further shown that primers corresponding to GSTs can be used to convert these tags into their corresponding longer end fragments. The GST technique can be used, with minor modifications, for SAGE analysis of eukaryotic mRNAs and might be adaptable for profiling gene expression in prokaryotes.

### BRIEF DESCRIPTION OF THE DRAWINGS

FIG. 1 is schematic representation of the GST protocol. In this method, DNA is first fragmented with a rare cutter such as NotI, or a more frequent cutter such as BamHI. Specific complementary biotinylated linkers are ligated to the free ends, and the DNA is then digested with NlaIII. All subsequent steps in the protocol are identical.

FIG. 2 represents length distribution of Y. pestis BamHI-NlaIII GSTs. The number of GSTs is plotted on the Y axis. Their lengths are plotted on the X axis. Shown in green are the predicted GSTs, in blue are the observed GSTs, and in red are the unseen GSTs.

### DETAILED DESCRIPTION OF THE INVENTION

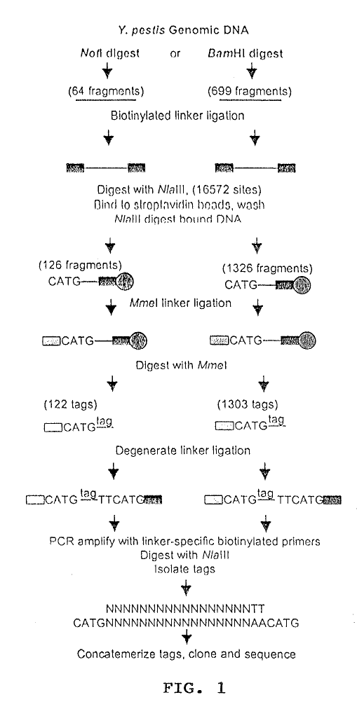

FIG. 1 gives the general strategy for production of GSTs. The method depends on the ability of a type II restriction enzyme, termed the fragmenting enzyme, to cleave the starting DNA into a manageable number of fragments, all having the same complementary single-stranded extensions. The digest is then ligated with a molar excess of short biotinylated duplex complementary adapters with only one cohesive end, to biotinylate both ends of all the fragments. The DNA is next digested with NlaIII, the anchoring enzyme, which cleaves leaving four-base cohesive ends. Biotinylated end fragments are recovered by binding to streptavidin-coated magnetic beads and digested a second time with NlaIII to assure that NlaIII digestion is complete. After the beads are washed, a duplex linker with NlaIII cohesive termini is ligated to the bound DNA fragments. This linker generates a recognition site (TCCGAC) for the type IIS enzyme MmeI, the tagging enzyme, only when it is joined to NlaIII cohesive ends.

After washing to remove excess linkers, the beads are incubated with MmeI to release the linker and appended tags from the beads. Since the last C residue in the adapter's MmeI recognition site partially overlaps the NlaIII site of the bound DNA, the released fragments contain 21 bases of sequence information from the starting DNA. These products are recovered and ligated with an adapter with a 16-fold degenerate 3′ overhang (Spinella et al., Nucleic Acids Res 27: e22 (1999)) which renders it compatible with all possible two-base 3′ overhangs released by MmeI. This adapter was designed to add two consecutive T residues and a second NlaIII site on the ends of the original MmeI generated fragments (TTCATG . . . ). The ligation products are PCR-amplified using two linker-specific biotinylated primers, cleaved with NlaIII and the two biotinylated end fragments removed by affinity capture on streptavidin-coated magnetic beads (Powell et al., Nucleic Acids Res 26: 3445-6 (1998)) leaving the 19-base pair duplex GSTs with NlaIII cohesive ends tags free in solution (FIG. 1). Each tag ends with two T/A base-pairs donated by the degenerate linker which help stabilize the identifier portion of the tag. They also act as a punctuation sequence to demarcate individual tags and aid in determining their polarity. The purified tag fragments are ligated together to form concatemers. Concatemers of sufficient minimal length are isolated by agarose gel electrophoresis and ligated into a pZero-based positive selection vector. The recombinant plasmids are electroporated into competent E. coli cells to generate the GST library in preparation for DNA sequence analysis.

In developing the GST method, it was reasoned that adapter ligation would be more specific than enzymatically filling in the cohesive ends with biotinylated nucleotides. This might be especially important in cases where obtaining nearly intact starting DNA is problematic. An additional benefit of adding a linker to the fragmented DNA is that it helps avert steric hindrance during the subsequent enzymatic reactions that are performed once the DNA is captured on magnetic beads.

A critical step contributing to the robustness of the GST protocol is the amount of material produced during the first round of PCR amplification. Typically, when this reaction is analyzed by electrophoresis on a 10% polyacrylamide gel, a band with the expected mobility of the GSTs plus attached linker arms, 94 bp, is observed, plus varying amounts of diffuse material with slower mobilities. The amount of this diffuse material in the reaction seemed to be proportional to the number of PCR amplification cycles; therefore, it was reasoned that it most probably represents amplicon heteroduplexes, formed by preferential perfect annealing of the low complexity linker arms, but imperfect annealing of the internal tags at high product concentrations. As expected, the bulk of this material is sensitive to digestion with S1 nuclease (data not shown). To optimize amplicon recovery, a linear amplification step was introduced to reduce heterogeneity (LARHD) which uses one extra round of amplification to convert the bulk of the reaction products to double-stranded DNA. Several additional tests showed that the potential to form heteroduplexes could be avoided during additional rounds of PCR amplification of the LARHD products by doing repeated rounds of linear amplification with one GST linker-specific primer followed by one final amplification step after addition of the second linker specific primer. Unwanted PCR primers that would be carried over from the LARHD step are eliminated by incubation with Exo I, which preferentially hydrolyzes any remaining single-stranded primers (Hanke et al., Biotechniques 17: 858-60 (1994)). Digestion with Exo I is also used to solubilize any free primers after the final amplification steps, prior to digestion with NlaIII to release the internal identifier tags from their flanking GST linker cassettes. Since the linker-specific primers used in amplification are biotinylated at their 5′ end, streptavidin beads can be used to capture the liberated cassettes, thereby avoiding losses that would accompany gel purification of the 19-bp long tags (Powell et al., Nucleic Acids Res 26: 3445-6 (1998)).

Disclosed herein is a method for obtaining 21-22 bp Genomic Signature Tags from predetermined positions in genomic DNAs. In principle, the method can provide limited representation of all the DNA molecules in a sample without prior knowledge of the DNA sequence. The approach can be fine-tuned by the user to provide different degrees of coverage and discriminatory power. The method is similar to the TALEST protocol (Spinella et al., Nucleic Acids Res 27: e22 (1999)) in that it utilizes a 16-fold degenerate linker cassette to attach known sequences to the ends of MmeI digested DNAs, thereby taking advantage of being able to use cohesive termini for high-efficiency linker addition. Addition of this linker provides not only an appended sequence for PCR amplification, but it also attempts to reduce biases during amplification by flanking the GSTs on both sides with distinct, long linkers. Since the degenerate linker is in molar excess during ligation to the MmeI generated ends, few tags should self-ligate and be sandwiched by the same GST linker. GST panhandle structures, which would result in low amplification efficiency, are thereby avoided. In contrast, excess degenerate linker, which should dimerize during ligation, is expected to form panhandles that should suppress their amplification. Other nonstandard steps in the GST amplification strategy include two separate rounds of linear amplification to generate sufficient material for library construction, while at the same time reducing product heterozygosity.

In order to test and validate the quantitative precision of the GST approach, a BamHI-NlaIII library was produced from Y. pestis DNA, as described below. Tags were produced, concatenated, and purified by agarose gel electrophoresis, and cloned into a positive selection pZero vector. To increase the efficiency of cloning longer inserts, a two step ligation strategy was used. (Damak et al., Biotechniques 15: 448-50, 452 (1993)). Initially, an excess of GSTs is ligated with the SphI digested vector at a high DNA concentration, a condition which promotes further concatemerization of the tags onto ends of the linearized vector. The reaction is then diluted and incubated overnight under conditions that favor circularization. The resulting clones typically contained 20 to ≧40 GSTs.

The results of this study show that the GST technique provides a route to obtaining numerous 21-22 bp sequence tags that can be used to identify the DNA source, and as shown here, the presence or absence of particular tags can provide some indication of the genetic variability between two closely related strains The length of the tags allow direct determination of the source DNA if the sequence is available. In silico comparison of all the BamHI-NlaIII GSTs that would be generated from a mixture of the 60 complete microbial genomes, the NCBI database demonstrated that these different bacterial strains share few GSTs in common. Table 4 contains a list of the top 30 shared tags. The worst case scenario is the occurrence of a single tag that was found three times in E. coli and once in Y. pestis. No GST was shared by three strains, although this might change as more closely related organisms are sequenced. Even between closely related strains, the frequency of unique, unshared identifiers is more than adequate to allow strain differentiation. A comparison between the 4.6 Mb E. coli K12 and 5.5 Mb O157H7 genomes predicts that they would generate 863 and 1018 unique BamHI-NlaIII GSTs, respectively. While they share 554 common tags which would classify the DNA as being E. coli, the K12 genome has 309 unique GSTs, and the 0157H7 genome has 464 that might be used to accurately differentiate between them.

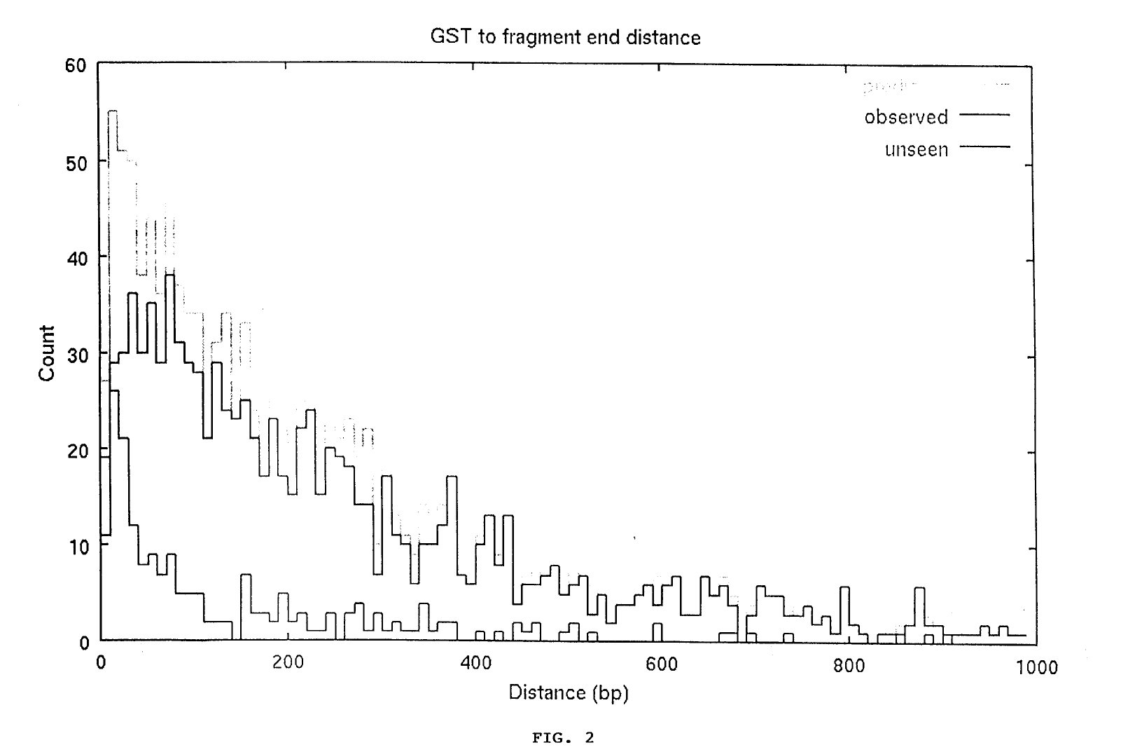

Assuming a 50% G+C content, an enzyme such as NotI with an 8-base recognition sequence will cleave on average every 48 (65.5 kb) bases compared to every 46 (4 kb) bases for a restriction enzyme with a 6-base recognition sequence, such as BamHI. In practice, this means that fragmenting the DNA with BamHI will usually produce 10× more GST tags from a genome, than would fragmentation with NotI. Other factors that influence the average fragment size generated by the fragmenting enzyme are: G+C content, dinucleotide frequency, and sensitivity to methylation. CpG methylation completely blocks cleavage by NotI, and such sites would be missed if only NotI were used for fragmentation. Fortunately, there are at least 10 other commercially available enzymes with specificities greater than 6 bases that can be used for GST fragmentation. Some of these enzymes, such as PacI (recognition sequence TTAATTAA), cut only A+T rich DNAs while others cut mainly G+C rich DNAs, but are not sensitive to CpG methylation. Application of GST to analyze complex mixtures of prokaryotic and eukaryotic DNAs may necessitate the use of two or more fragmentation enzymes to ensure an adequate depth of GST coverage. In choosing a fragmentation enzyme, using ones that leave cohesive ends for ligation with appropriate biotinylated linker cassettes is preferred. It is believed that cohesive end mediated ligation with a biotinylated linker cassette is an important discriminatory GST tool as it alleviates the problem of having to enzymatically biotinylate only the ends of the DNA that were generated by enzymatic cleavage, which in practice can be very difficult when dealing with DNA isolated from non-laboratory sources where degradation may be a problem. In fact, for GST analysis the starting DNA does not have to be high molecular weight since as shown in FIG. 2, even a relatively small fragment containing a site for the fragmenting enzyme should carry a nearby site for the NlaIII tagging enzyme.

The only mathematical assumption behind the GST method is that the probability of observing specific GSTs should closely follow the Poisson distribution. Therefore, the probability of observing a given tag with 1/N abundance while sequencing N tags is 0.63. Tags with abundance larger than 1/N should be sampled more frequently, provided that the PCR amplification and subsequent cloning steps used to obtain the library are not biased, which would compromise the quantitative aspects of the method. In developing the GST method, several steps were critically evaluated to help ensure that the frequency of tags in the library reflects the frequency of tags in the genomic DNA from which the tags were derived, The frequency distribution of the tags in the Y. pestis database appears to be quite flat, and as might be expected, many of the most abundant GSTs were derived from repetitive sequences. This means that the technique should, in addition to being able to identify DNA sources, be able to provide a fairly accurate means for quantitative analysis of mixed DNA populations. This concept is currently being tested by preparing a NotI-NlaIII GST library using DNA isolated from a non-stoichiometric mixture of five different bacteria strains.

The data show that 21 bp GSTs can be used efficiently as primers to PCR amplify the DNA between specific tagging and fragmenting sites. The sequence of the products can then be used to provide more information for deeper phylogenetic analysis of genomic samples, or as hybridization probes to facilitate isolation of complementary clones from whole genome libraries. Since GST analysis is a direct PCR-based DNA sequencing approach for profiling DNA, it could be applied to analyze DNA composition in complex mixtures, and it could circumvent the need to isolate and grow organisms for measurement of microbial diversity and distribution in natural communities. This information could be used in conjunction with traditional cultural techniques, to help complete the catalogue of species present in a sample.

Only minor changes in the GST protocol are needed to use the method for classical SAGE analysis of poly (A)⁺ eukaryotic mRNAs. In this case, double-stranded cDNA is synthesized from the mRNA by means of a biotinylated oligo (dT) primer anchored to streptavidin beads (Virlon et al., Proc Natl Acad Sci U S A 96: 15286-91 (1999)). The cDNA is then cleaved with NlaIII leaving the 3′ most portion of the cleaved cDNA with the cohesive overhang needed for ligation of the MmeI adapter. All other steps then proceed as outlined in FIG. 1. This method has been used to obtain 21-22 bp SAGE tags to profile gene expression in human platelets and in Pfiesteria piscicida, a toxic dinoflagellate. It should also be possible to modify the GST method to profile prokaryotic gene expression by using biotinylated oligonucleotides to remove the bulk of the 16S and 23S rRNA in total bacterial RNA samples. A commercial kit based on this principle which is purported to be suitable for mRNA purification from a broad spectrum of Gram-positive and Gram-negative bacteria has been recently introduced by Ambion. One approach would be to convert the purified bacterial mRNA into cDNA using random hexamer priming and reverse transcriptase. The cDNA could then be used to generate cGSTs to profile the expressed regions of the genome. As an illustrative example, the NCBI database lists 3885 genes in the chromosome of Y. pestis of which 664 encompass one or more complete GSTs in a BamHI-NlaIII library. The 765 GSTs from a EcoRI-NlaIII library would sample an additional 566 coding regions.

In summary, the basic GST procedure provides a means for genome-wide fingerprinting of chromosomal and episomal DNAs, and by extension, for compositional analysis of natural populations. It can be performed with equipment available in most molecular biology laboratories. With a few modifications, the method can be used as a tool for profiling gene expression. The length of the tags is sufficient for recognizing, with BlastX, potential 7-amino acid sequences from proteins that may be of interest. These regions of DNA can then be targeted for synthesis of longer fragments for gene identification and possible expression. The tags themselves can also be used to determine nucleotide motif frequencies. In addition, statistical data such as GC content, dinuclectide relative abundance, and overlapping 9 bp motifs, can be derived from the 21-22 bp long tags. These parameters could serve as classification tools in biodiversity studies (Karlin et al., Trends Genet 11: 283-90 (1995); Sandberg et al., Genome Res 11:1404-9 (2001))

### EXEMPLIFICATION

Analysis of a Y. pestis BamHI GST Library

Shown in FIG. 1 and Table 1 are the predicted numbers of tags which would be generated at each step of the procedure from Y. pestis DNA, using either NotI or BamHI as the fragmenting enzyme. Using the 4.7 Mb, Y. pestis CO92 complete genome (minus the pCD1 plasmid) as input (Parkhill et al., Nature 413: 523-7 (2001)), it was determined in silico that there should be 64 cleavage sites for NotI, 699 sites for BamHI, and 16,572 sites for NlaIII. Only one NotI fragment is predicted to lack an internal NlaIII site, but 36 of the smaller fragments generated by BamHI should not be cleaved by NlaIII. The mean lengths of the resulting NotI-NlaIII and BamHI-NlaIII fragments are 273 and 267 bp, respectively. The similarity in these mean fragment lengths reflects both the high density and nearly random distribution of NlaIII sites in the Y. pestis genome. Only 11 of the NotI-NlaIII and 90 of the BamHI-NlaIII fragments are predicted to be less than 21 bp long, all other fragments should generate full-length 21 bp tags. If only 21 bp tags are considered, then the NotI-NlaIII library should sample about 2.4 kb of the Y. pestis sequence, while the BamHI-NlaIII library would sample about 10 times more DNA, about 26 kb.

One problem that is intrinsic to the method, occurs when the MmeI recognition sequence (GTYGGA) is within 21 bp of the NlaIII end. This sequence would direct cleavage back towards the NlaIII end allowing MmeI to potentially cut within the attached MmeI linker which would interfere with subsequent PCR amplification. A GTYGGA sequence within the next 21 bp could potentially give rise to tags less than 21 bp long depending upon which site is first recognized by MmeI. Analysis of the Y. pestis sequence indicates that MmeI digestion would at most eliminate only 17 tags from a BamHI library, but none from the NotI-derived library. While all of the 21 bp NotI derived tags are unique, 47 of the BamHI derived 21 bp tags come from 14 repeated sequences, and therefore occur two or more times within the database.

To validate the generality of this method, a Y. pestis GST library was prepared using BamHI as the fragmenting enzyme since it will generate sufficient tags for meaningful data analysis. Sequence analysis of the initial library showed that MmeI can liberate both 21 and 22 bp long tags from the same location in the DNA. Analysis of this library, which was prepared using a single NlaIII digestion step, also revealed the presence of a large fraction of tags originated from NlaIII sites that were not proximal to a BamHI site. The presence of these tags in the library obviously was the result of incomplete NlaIII digestion. Therefore, a second NlaIII digestion step is now routinely included after the biotinylated fragments are captured on the magnetic beads in order to obtain more complete digests. The data reported here are from a single library prepared following the steps outlined in FIG. 1. The cloned inserts in this library were typically several hundred bp to slightly less than one kb long.

The linker used to biotinylate the BamHI digest adds 12 bp to the ends of each fragment. In principle, the addition of this linker should allow MmeI to liberate 21 bp long tags even from the 90 BamHI-NlaIII fragments that are less than 21 bp long. In these cases, MmeI would have to cleave within the attached linker. Tags from these sites are easy to identify as they should contain a BamHI recognition sequence near their 3′ ends. To simplify discussion, fragments are numbered according to their order along the DNA and use R (reverse) and F (forward) to indicate the relative location of the GST within the fragment. Thus, R314 indicates the reverse GST from BamHI fragment number 314, which would be followed by F314 (the next forward GST), R315, F315, etc.

A total of 5,432 GSTs were extracted from the sequenced arrays. The number of 21 and 22 bp long tags was approximately equal, 2,701 and 2,731 respectively. The vast majority, 5,268 (97%), exactly matched at 1,133 sites in the Y. pestis genome. This includes a total of 336 tags which were uniquely matched at 88 correct tagging sites, even though their initial polarities were ambiguous. Most of these unique matches could be assigned to the first NlaIII site next to a BamHI fragmentation site, which indicates that the two step NlaIII digestion was virtually complete. Only 59 (1%) of the extracted tags exactly matched interior NlaIII sites. These tags could result from over-digestion with BamHI or partial NlaIII digestion. However, it is thought that several may have arisen because subtle changes in the genome introduced new BamHI sites. This seems to be the case for fragments 90 and 459, which each gave rise to two internal tags. Two other internal tags occurred twice, which because of the large number of total NlaIII sites in the Y. pestis DNA, is a highly improbable random event. A small number of tags (6) that passed all of the editing criteria, have no obvious close match to the Y. pestis genome or any other sequence in GenBank. These might originate from sequences that are unique to the EV766 genome or represent spurious tags generated during library construction, amplification, and cloning. Of the total predicted potential tagging sites, 209 were still unseen. It is believed that many, but not all, of these unseen sites would be matched if the sample size were increased (see below). A detailed analysis of the data is available at URL (in preparation).

To a first approximation, cloning and sequencing of GSTs should be random processes, and on average, the relative frequency of occurrence of a particular GST in a library should reflect its frequency in the DNA sample. Therefore, tags from highly repetitive regions of the chromosome, or from higher copy number plasmids, should be more numerous than tags from unique regions. This prediction seems to hold true for the GST library. As shown in Table 2, the most numerous tag encountered is the one predicted to occur most frequently (8 times) in the Y. pestis chromosome. It was followed in order by the tag predicted to be the next most frequent, the one occurring 7 times. Only one tag should be present 5 times, one 4 times, three tags should each be found three times, and seven tags should each occur twice. Two other redundant tags listed in Table 2 should not be recovered at all since each contains a BamHI fragmentation site very close to its 5′ end. The actual observed frequency of the multiple tags is highly correlated (r=0.88) with the predicted frequency. However, one tag that is predicted to be present 4 times in the genome seems to be under represented in the database. This tag is associated with an IS100 element that is known to be a source for genetic variability in different Y. pestis isolates (Motin et al., J Bacteriol 184: 1019-27 (2002)), which may in part explain these results. The two plasmids, pMT1 and pPCP1, thought to be present in the EV766 genome, each contain a single BamHI site and each should have contributed two unique tags to the library. All four tags were catalogued at about the same frequency as single-copy chromosomal tags. This would suggest that neither of these plasmids had a significantly elevated copy number in the strain used here, a prediction that was confirmed by inspection of agarose gel profiles of the total genomic DNA used for this study.

Such deviations in tag frequency or occurrence can also occur when sequence changes introduce or remove a fragmenting site or tagging site. Loss or gain of a single fragmenting site will at most affect the two GSTs flanking the site. Deletions or insertions on the other hand can simultaneously remove or add several tags. Analysis of the data for the absence of adjacent tags revealed several places where deletions must have occurred in the EV766 genome. The most striking example is the failure to recover any of the expected 25 consecutive tags from a segment beginning with F314 and ending with F327 (bp 2,172,627 through 2,254,447 if the 3′ position of BamHI site 327 is included). This region contains a 37 kb high-pathogenicity island encoding virulence genes involved in iron acquisition from the host via a siderophore called yersiniabactin (the ybt biosynthetic gene cluster) (Buchrieser et al., Infect Immun 67: 4851-61 (1999)). It is part of a larger, 100 kb region termed the pgm (pigmentation) locus. This locus can delete spontaneously, probably by homologous recombination between its two flanking IS100 elements (Fetherston et al., Mol Microbiol 6: 2693-704 (1992)). Such a deletion would eliminate tags F314-F327; therefore, it is proposed that strain EV766 lacks the entire pgm locus. Similar analysis also identifies a potential deletion of the region bounded by R194-R197, which normally harbors an IS1541 insertion element. Deletions or other changes may have eliminated tags F237-F238, another region associated with an IS100 element. Several other regions not associated with known IS elements that also seem to have been deleted or undergone DNA rearrangements that eliminate consecutive tags are listed in Table 3. If these 44 tags are excluded, the number of unseen tags drops to 144.

A small fraction of catalogued tags, totaling 164 (3%), appears to contain point mutations. Inspection of the relevant single-pass sequencing chromatograms indicates that the original base calls were accurate. In nearly every case, the corresponding correct GST could be found in the data set. Presumably these differences represent errors introduced during library preparation, rather than true polymorphisms in the DNA sample. The distribution of mismatches within the tags was not totally random; discrepancies were somewhat more frequent within the last two bases at the 3′ end of the tag. This most likely reflects mis-ligation between the MmeI overhangs and the 16-fold degenerate cassette during this step in the GST protocol. Increased fidelity should be possible by using a lower concentration of the degenerate adapter, shorter incubation times, or higher temperature during the ligation step. One empirical way to eliminate most of these errors is to omit tags encountered only once from further analysis, as is typically done to help eliminate sequencing and other errors from SAGE libraries. This type of filtering would eliminate all but 23 of the imperfectly matched tags from further consideration.

The sequence complexity and length of a GST, 21-22 bp, should in most cases be sufficient to enable its use directly as a primer to amplify the stretch of DNA between the tagging site and the proximal site for the fragmenting enzyme. To test this concept, a group of five tags predicted to begin approximately 100 to 1000 bp away from their proximal BamHI sites were selected and used for custom primer synthesis. Template Y. pestis DNA was digested with BamHI and ligated with a linker cassette that introduced an identical priming site at the both ends of each fragment. The DNA was then digested with NlaIII to physically separate the Tinkered BamHI ends. Aliquots were then subjected to ten rounds of linear PCR amplification using just the GST-specific primer to increase the amount of complementary single-stranded targets in the sample. This step was then followed by twenty-five PCR cycles with both primers. Each reaction generated a distinct band of the expected length. Direct sequencing of these five bands unequivocally confirmed their correct location in the Y. pestis genome.

While the data obtained show that desired objectives were obtained, further analysis (FIG. 2) suggests that under sampling tags that lie a short distance from the fragmenting site may be occurring. This deficiency can be easily addressed by increasing the length of the biotinylated cassette used to attach the DNA to the streptavidin beads. In this context it is worth noting that Wang et al. (P-roc Natl Acad Sci U S A 95:11909-94 (1998)) observed a SphI site (GCATGC) tethered to a streptavidin bead by a short linker could be cut with SphI, but not by NlaIII, even though the linker contained a CATG sequence.

Methods

DNA Fragmentation and Biotinylated Adapter Ligation

DNA from avirulent Yersinia pestis EV766, a Ca²⁺ independent strain cured of the 70.5 kb pCD1 plasmid but retaining the pPCP1 9.5 kb and 100 kb pMT1 plasmids (Portnoy et al., J Bacteriol 148: 877-83 (1981)), was kindly provided by James Bliska, SUNY-SB. Ten micrograms was digested with 100 U of BamHI (New England Biolabs, NEB, Tozer, Mass.), extracted with an equal volume of phenol/chloroform (P/C), and precipitated with ethanol. After centrifugation, the pellet was resuspended in 50 l TEsl (10 mM Tris-HCl, pH 8.0, 0.1 mM EDTA-Na₃). A biotinylated GATC oligonucleotide adapter cassette was created by mixing 7200 pmol each of two synthetic oligonucleotides (sense strand: CGA ACC CCT TCG; antisense strand: P-GAT CCG AAG GGG TTC GT-BIOTIN in 100 l OFA buffer (10 mM Tris-acetate, pH 7.5, 10 mM Mg acetate, 50 mM K acetate, Amersham Bioscience, Piscataway, N.J.)) heating them to 95° C. for 2 min and then allowing them to cool slowly to room temperature. An approximate 50-fold excess of biotinylated cassette (600 pmol), relative to available BamHI ends, was ligated to the fragmented DNA in a total volume of 50 μl of 1× ligase buffer (Takara) containing 350 U of T4 DNA ligase (Takara). The reaction was incubated overnight at 16° C. followed by extraction with an equal volume of P/C The sample was precipitated with ethanol, centrifuged and resuspended in 50 μl TEsl.

First Digestion with NlaIII and Binding to Magnetic Beads

The fragmented DNA was next digested with 25 U of NlaIII (NEB) in 100 μl NlaIII digestion buffer (1× NEB buffer #4 supplemented with 1×BSA and 10 mM spermidine HCl₃ for 4 h at 37° C.; NlaIII digestion is stimulated 2 to 4-fold by addition of spermidine, unpublished observations). One hundred μl (1 mg) of streptavidin magnetic beads (Dynal Biotech. Inc., Lake Success, N.Y.) were washed twice with 200 μl of 1× magnetic bead binding buffer (MBB: 10 mM Tris-HCl, pH 7.4, 1 mM EDTANa₃, 1 M NaCl), and then resuspended in 100 μl of 2×MBB. The beads were then added to the NlaIII digested DNA in a non-stick 1.5 ml microfuge tube (Ambion, Austin, Tex.). The beads and digest were mixed gently for 1 h at room temperature to bind biotinylated BamHI-NlaIII fragments.

Second Digestion with NlaIII and MmeI Adapter Ligation

A second incubation with NlaIII was performed on the bound fragments by resuspending the beads in 200 μl NlaIII digestion buffer containing 25 U of enzyme and incubating for 2 h at 37° C. The beads were washed three times with 200 μl TEsl to remove non-bound DNA fragments and one time with 200 μl 1×T4 ligase buffer. A MmeI oligonucleotide adapter was created by mixing and annealing, as described above, 1000 pmol each of two synthetic oligonucleotides (sense strand: TTT GGA TTT GCT GGT CGA GTA CAA CTA GGC TTA ATC CGA CAT G: antisense strand: P-TCG GAT TAA GCC TAG TTG TAC TCG ACC AGCA AAT) CC-AmMC7 in 100 μl 1×OFA. The annealed MmeI adapter cassette (40 pmol) was ligated to the fragmented solid-phase DNA for 2 h at 16° C. in a total volume of 50 μl of 1× ligase buffer (Takara) containing 350 U of T4 DNA ligase (Takara).

Digestion with MmeI

Beads were washed six times with 400 μl 1×MBB and then washed several times with 200 μl MmeI digestion buffer (100 mM HEPES, pH 8.0, 25 mM K acetate, pH 8.0, 50 mM Mg acetate, pH 8.0, 20 mM DTT, 4 mM S-adenosylomethionine-HCl). The beads were then resuspended in 100 μl 1×MmeI digestion buffer containing 8 U MmeI (Center of Technology Transfer, Gdansk, Poland) and incubated for 2 h at 37° C. with occasional mixing. The beads were collected and the supernatant containing the released tags was removed to a clean microfuge tube. The beads were washed with 100 μl TEsl and the wash combined with the first MmeI supernatant. The pooled MmeI digest is extracted with an equal volume of P/C and precipitated at −80° C. for 1-2 h with 1 ml of ethanol after addition of 133 μl 7.5 M ammonium acetate and 2 μl Glyco Blue (Ambion) as carrier. The resulting pellet was washed with cold 75% ethanol, dried in vacuo and resuspended in 29.5 μl TEsl plus 4 μl 10×T4 DNA ligase buffer.

Second Cassette Ligation and Initial PCR Amplification

A second, 16-fold degenerate adapter cassette was prepared by annealing two synthetic oligonucleotides as described above (sense strand: P-TTC ATG GCG GAG ACG TCC GCC ACT AGT GTC GCA ACT GAC TA-AmMC7; antisense strand: TAG TCA GTT GCG ACA CTA GTG GCG GAC GTC TCC GCC ATG AAN N). Thirty-five pmol of adapter cassette (3.5 μl) was added to the resuspended tags, and after 15 min at room temperature, 3 μl of ligase (1000 U-Takara) was added and the reaction incubated overnight at 16° C. The ligation products were subjected to PCR amplification consisting of an initial denaturation step at 95° C. for 2 min followed by 35 cycles of 95° C. for 30 s, 58° C. for 30 s and 72° C. for 30 s with a final extension step at 72° C. for 4 m using 5′-Biotin-GGA TTT GCT GGT CGA GTA CA and 5′-Biotin-TAG TCA GTTGCG ACA CTA GTG GC as forward and reverse primers, respectively, each at a final concentration of 0.1 μM. Cycling was performed in 1× Promega buffer containing 3 mM Mg sulfate and 20 pM of each dNTP. Typically, 1.0 μl of ligation product was amplified in a 200 μl reaction containing 1 μl Platinum Taq DNA polymerase mixture (Invitrogen).

Linear Amplification to Reduce Heterogeneity (LARHD)

The PCR products were then subjected to one round of linear amplification to reduce heterozygosity (LARHD) by diluting them to 1 ml with 800 μl 1×PCR buffer containing 4 μl Platinum Taq and 50 pmol of each Biotinylated primer. The reaction was then incubated at 95° C. for 2.5 m, 58° C. for 30 s, and 72° C. for 5 m. Unincorporated primers were digested by addition of 10 μl (200 U) of single-strand specific E. coli Exo I. After 1 h at 37° C. the sample was P/C extracted and precipitated by addition of 2.5 volumes of ethanol in the presence of 0.3 M Na acetate, pH 6.0.

Second Linear Amplification (LARHD2), NlaIII Digestion and Concatemerization

Following centrifugation, the pellet was washed in 70% ethanol, dried and then dissolved in 200 ul TEsl. A portion (1/25th) was subjected to 25 additional rounds of linear amplification under the above LARHD conditions, except only the forward primer was added. This was then followed by one round of amplification after addition of the reverse primer and additional DNA polymerase to convert the linear amplification products to double-stranded DNA. Typically, 1 ml of sample is amplified and any unincorporated primers are hydrolyzed by incubation with Exo I as above. After P/C extraction and ethanol precipitation, the amplified DNA is digested with 20 U of NlaIII in 300 μl at 37° C. for 4 h (after 2 h the completion of digestion is checked by electrophoresis of a small aliquot on a 10% polyacrylamide gel). The digest is then extracted on ice with chilled P/C to prevent denaturation of the smaller GSTs and ethanol precipitated from Na acetate in the presence of Glyco Blue carrier. The sample is chilled for several h and then centrifuged at 4° C. The pellets are resuspended in 200 ul ice cold TEsl plus 25 mM NaCl, diluted with an equal volume of 2×MBB and added to 200 μl (2 mg) of streptavidin beads equilibrated with 1×MBB. After gentle mixing for 15 m at room temperature, the unbound fraction is transferred to a second 200 μl aliquot of beads to capture any remaining biotinylated DNA fragments. The unbound GST fraction is recovered and precipitated by addition of 2.5 volume of ethanol and Glyco Blue carrier and concatemerized with T4 DNA ligase (5 U/μl, Invitrogen) at 16° C. for 6 hr. The sample was subjected to electrophoresis on a 0.75% low melt agarose gel and products greater than 100 bp were recovered. These products were cloned into the SphI-site of a pZero plasmid (Invitrogen) that was engineered to have a SphI-minus KanR gene (unpublished). Recombinant clones obtained after electroporation of competent TOP10 cells (Invitrogen, Carlsbad, Calif.) are selected on 2×YT plates containing 50 μg/ml kanamycin. A schematic representation of the method is shown in FIG. 1 and a complete description of all steps is available at the web site (in preparation).

DNA Sequencing

Plasmid DNA for sequencing was prepared using Edge BioSystems reagents and protocols in 96-well plates. Templates were cycle sequenced using ABI Prism BigDye® terminator chemistry from the M13 forward primer and analyzed on ABI 3700 sequencers. Extracted data were ported to an Oracle® database and searched for valid tags using the GST software. The software ensures that only unambiguous 21-22 bp tag sequences, see below, are extracted for further analysis (tags with Ns, lengths other than 21-22 bases or whose polarity is unambiguous), are extracted to separate files for manual editing or further examination.

Ligation-Mediated PCR

Five Y. pestis-specific primers were synthesized: [535,384] CAT GCA GGG TGC ACG ACC CGA (205R); [2,281,342] CAT GTG GCC GCC GCG CTT AA (384R); [2,894,318] CAT GAC TCT GCC ATA GCT TCG (1031R); [3,452,611] CAT GCA GGA CCG CGG ACA ATG (102F); and [4,145,945] CAT GCA GTG CCA TCC TCA CGG (230F). The values in brackets are the position of the NlaIII tagging site in the Y. pestis chromosome. The values in parentheses are the distances between the respective NlaIII and BamHI sites, and the directionality of the PCR reaction. BamHI digested Y. pestis DNA was ligated with a non-biotinylated GATC oligonucleotide adapter created by mixing and annealing 1000 pmol each of two synthetic oligonucleotides (sense strand: CGT AAT ACG ACT CAC TAT AGG GA; antisense strand: GCA TTA TGC TGA GTT ATA TCC CTC TAG) in 100 μl OFA, as described above. The annealed GATC adapter (40 pmol) was ligated to BamHI fragmented DNA for 2 h at 16° C. in a total volume of 50 μl of 1× ligase buffer (Takara), containing 350 U of T4 DNA ligase (Takara). Aliquots of the Tinkered DNA were incubated at 94° C. for 2 min, followed by 10 rounds of linear amplification (94° C. for 20 sec, 55° C. for 30 s and 68° C. for 4 min) with the above Y. pestis-specific primers. This was followed by 25 additional rounds of amplification under the same conditions after addition of the common GATC-specific primer, the GATC sense strand. Products were extended for 10 m at 68° C. and analyzed on 6% polyacrylamide gels. Extension with the sense strand primer should add an additional 23 bp to the BamHI end of all the amplification products.

TABLE 1
Predicted GRIDS IDentifier tags for Y. pestis EV766

| | NotI fragmentation | | BamHI fragmentation | |
|---|---|---|---|---|
| | [64 fragments] | | [699 fragments] | |
| | | after MmeI | | after MmeI |
| | start^{(a)} | digestion | start^{(a)} | digestion |
| Tags of length ≧21 | | | | |
| predicted tags | 115 (7) | 115 (7) | 1236 (96) | 1214 (93) |
| unique tags | 115 (7) | 115 (7) | 1203 (94) | 1131 (91) |
| single tags | 115 (7) | 115 (7) | 1189 (92) | 1167 (89) |
| multiple tags | 0 | 0 | 14 (2) | 14 (2) |
| Tags of length ≦20 | | | | |
| predicted tags | 7 (0) | 7 (0) | 89 (12) | 89 (12) |
| unique tags | 7 (0) | 7 (0) | 86 (12) | 86 (12) |
| single tags | 7 (0) | 7 (0) | 84 (12) | 84 (12) |
| multiple tags | 0 | 0 | 2 (0) | 0 |
| zero length tags^{(b)} | 4 | 4 | 1 | 1 |
| SUM | 126 (7) | 126 (7) | 1326 (108) | 1303 (105) |

^{(a)}Values in parenthesis are the numbers of tags with ambiguous directions, i.e., they begin with the sequence CATGAA.
^{(b)}Zero length tags occur when the fragmenting site is immediately adjacent to a NlaIII site

TABLE 2
Corrcspondence Between Predicted and
Actual IDentifier Tag Frequencies

| | FREQUENCY | |
|---|---|---|
| IDentifier tag sequence^{(a)} | predicted | actual |
| ATCTGGAGGTTCCGTTC | 8 | 65 |
| CGTCATCTCGCTGAACG | 7 | 45 |
| GATGTATTTACGGCGTC | 5 | 34 |
| CCCTGCGGTACGGGAGC | 3 | 34 |
| CCTGCATTGGCACCGTT | 2 | 23 |
| CCAGCATCAGCCAGCCC | 2 | 22 |
| TAGGCTCGAGCCGCGCC | 3 | 20 |
| TCGTTCAAATCAAAGGA | 4 | 13 |
| CTGATAAACCGCGATCC | 2 | 13 |
| AATCCTCACCTAACCGA | 2 | 12 |
| CTTTCGTTGGTTAGCCA | 3 | 11 |
| CCCCAGCCCTGGCCCGC | 2 | 11 |
| AACCGCGTATCAATCAG | 2 | 11 |
| TGCGTTTTCAGGACCGT | 2 | 9 |
| TTGGATCCGAAGGGGTT | 3 | unseen-contains BamHI site |
| GGGATCCGAAGGGGTTC | 2 | unseen-contains BamHI site |

Complete lists of GSTs, in both order of abundance and position in the Y. pestis genome, are available via the internet at http:/www.
^{(a)}CATG omitted

TABLE 3
Potential Deletions in the Y. pestis EV766 genome

| Start-End | Position bp | IS Element | # of tags effected |
|---|---|---|---|
| F314-F327 | 2,172,627-2,254,447 | yes IS100 | 25 |
| R194-R197 | 1,307,243-1,316,087) | yes IS1541 | 7 |
| F227-F228 | 1,554,643-1,556,368 | no | 3 |
| F237-F238 | 1,618,033-1,652,133 | yes IS100 | 3 |
| F381-F382 | 2,662,263-2,685,036 | no | 3 |
| F453-F454. | 3,069,009-3,122,266 | no | 3 |
| Total | | | 44 |

TABLE 4
Shared GSTs Between Two Differcut Bacteria^{a}

| GST sequence^{(b)} | organisms | total | organism (count) | organism (count) |
|---|---|---|---|---|
| GCCGCTTAACCGCCGCA | 2 | 4 | Escherichia coli (3) | Yersinia pestis (1) |
| GATCGCCGATCGTCCCG | 2 | 3 | Mycobacterium leprae (1) | Mycobacterium tuberculosis (2) |
| GCAACGATATTGGTGAC | 2 | 3 | Mycobacterium leprae (1) | Mycobacterium tuberculosis (2) |
| CCGCCCCGGAAATCACC | 2 | 3 | Mycobacterium leprae (1) | Mycobacterium tuberculosis (2) |
| GACCTGTCCACCGGCAA | 2 | 3 | Mycobacterium leprae (1) | Mycobacterium tuberculosis (2) |
| GGCTGTGGGTGGCGTTC | 2 | 3 | Mycobacterium leprae (1) | Mycobacterium tuberculosis (2) |
| CTTGGCCGCTACACCAC | 2 | 3 | Pyrococcus abyssi (1) | Pyrococcus horikoshii (2) |
| CTCCGCCGCTTGTGCGG | 2 | 3 | Mycobacterium leprae (1) | Mycobacterium tuberculosis (2) |
| GTGGATGCCTTGGCATC | 2 | 3 | Mycobacterium leprae (1) | Mycobacterium tuberculosis (2) |
| GCGACCCAGGAACAGCA | 2 | 3 | Mycobacterium leprae (1) | Mycobacterium tuberculosis (2) |
| GGAGTCGATGTTATCGG | 2 | 3 | Mycobacterium leprae (1) | Mycobacterium tuberculosis (2) |
| AAGCCGGTCGCCATCAT | 2 | 2 | Mesorhizobium loti (1) | Sinorhizobiumme liloti (1) |
| GTGACTTCTGCGGATGT | 2 | 2 | Chlamydia muridarum (1) | Chlamydia trachomatis (1) |
| TGCACCGGAATGCGGAT | 2 | 2 | Mesorhizobium loti (1) | Sinorhizobiumme liloti (1) |
| CACCACCTCTCCTTCTA | 2 | 2 | Thermoplasma acicdophilum (1) | Thermoplasma volcanium (1) |
| TCGGACAGAACCTTGCG | 2 | 2 | Agrobacterium tumefaciens (1) | Sinorhizobium meliloti (1) |
| ACGCCGAAGGTGATGGC | 2 | 2 | Mesorhizobiumloti (1) | Sinorhizobiumme liloti (1) |
| AACGAAGATCAATTTCC | 2 | 2 | Chlamydia muridarum (1) | Chlamydia trachomatis (1) |
| AATTAGAAAATTATGAC | 2 | 2 | Haemophilus influenzae (1) | Pasteurella multocida (1) |
| CGGACTTCGGTCGGCTT | 2 | 2 | Mesorhizobiumloti (1) | Sinorhizobium meliloti (1) |
| CTCTCAACGTAGGGAAC | 2 | 2 | Pyrococcusabyssi (1) | Pyrococcushorikoshii (1) |
| CCCATCACTATCAAGCC | 2 | 2 | Chlamydiamuridarum (l) | Chlamydia trachomatis (1) |
| AGCAGGTTGAAGCTTCA | 2 | 2 | Mycoplasmagenitalium (1) | Mycoplasma pneumoniae (1) |
| ATGCGCAAGTGCCATCT | 2 | 2 | Agrobacterium lumefaciens (1) | Sinorhizobium meliloti (1) |
| CAGGTCGGCATTTAACC | 2 | 2 | Pyrococcus abyssi (1) | Pyrococcus horikoshii (1) |
| AAGGTTCAACGTGGGTC | 2 | 2 | Thermoplasma acidophilum (1) | Thermoplasma volcanium (1) |
| CGGGGAAACGTAGTACC | 2 | 2 | Chlamydia muridarum (1) | Chlamydia trachomatis (1) |
| CACAAGATCCAGGACCG | 2 | 2 | Mesorhizobium loti (1) | Sinorhizobium meliloti (1) |
| AGCTAACCCCATTTTGT | 2 | 2 | Chlamydiam uridarum (1) | Chlamydia trachomatis (1) |
| CAGCACTCCATATTTTA | 2 | 2 | Clostridium acetobutylicum (1) | Pyrococcus horikcoshii (1) |

^{(a)}GSTs within 25 bp of the BamHI fragmentation site were omitted;
^{(b)}CATG omitted

1. A method for generating a genome signature tag library, comprising:
a) providing a genomic DNA-containing sample;
b) contacting genomic DNA in the genomic DNA-containing sample with a type II restriction enzyme, under conditions appropriate for complete digestion of the genomic DNA by the type II restriction enzyme, thereby generating a plurality of digestion fragments, each digestion fragment having complementary cohesive termini;
c) incubating the digestion fragments of step b) with a molar excess of biotinylated duplex complementary adapter fragments, each having only one cohesive end, under conditions appropriate for ligating one biotinylated duplex complementary adapter fragment to each end of the digestion fragments of step b);
d) contacting the product of step c) with the restriction enzyme NlaIII, under conditions appropriate for complete digestion by the restriction enzyme;
e) recovering labelled fragments following the digestion of step d) by capture with streptavidin-coated magnetic beads;
f) incubating the magnetic beads bearing recovered labelled fragments of step e) with a molar excess of duplex linker having NlaIII cohesive termini, under conditions appropriate for ligation of complementary cohesive termini, thereby generating a recognition sequence for the restriction enzyme MmeI at any location in which the duplex linker is ligated to an NlaIII cohesive termini;
g) removing excess linkers from the incubation mixture of step f);
h) incubating the magnetic beads with bound recovered labelled fragments from step g) with the restriction enzyme MmeI under conditions appropriate for complete digestion of the ligation product of step f), thereby releasing a fragment comprising duplex linker of step f) and an appended genomic signature tag;
i) recovering the genomic signature tag-containing fragment of step h);
j) incubating the recovered fragment of step i) with a molar excess of adapter fragment, the adapter fragment having a 16-fold degenerate 31 overhang, the adapter fragment adding two consecutive T residues and a second NlaIII restriction enzyme recognition sequence following productive ligation to a free MmeI restriction enzyme-generated end, the incubation being carried out under conditions appropriate for ligation of the adapter fragment to any recovered fragment having a free MmeI restriction enzyme-generated end;
k) amplifying the ligation products of step j) with a pair of biotinylated primers specific for the duplex linker of step f) and the adapter fragment of step j);
l) incubating the amplification product of step k) with the restriction enzyme NlaIII under conditions appropriate for complete digestion of the amplification product by the restriction enzyme;
m) capturing biotinylated end fragments generated in step l) using streptavidin-coated magnetic beads leaving tag fragments, comprising 19-base pair duplex genomic signature tags with NlaIII cohesive end tags, free in solution;
n) isolating the tag fragments from step m);
o) ligating the isolated tag fragments of step n) to form concatemers;
p) isolating concatemers of sufficient minimum length;
q) cloning the concatemers of step p) in a vector; and
r) transforming prokaryotic cells with the cloned concatemers of step q) to generate a genome sequence tag library.
